(19) United States
(12) Patent Application Publication
(10) Pub. No.: US 2016/0022277 A1
(43) Pub. Date: Jan. 28, 2016

(54) DISPOSABLE VENOUS TOURNIQUETS WITH REMOVABLE INDICIA-BEARING PORTION AND METHODS OF USE

(71) Applicants: Edward A. EIKMAN, Tampa, FL (US); Adam D. EIKMAN, Tampa, FL (US); E. Allan EIKMAN, Tampa, FL (US)

(72) Inventors: Edward A. EIKMAN, Tampa, FL (US); Adam D. EIKMAN, Tampa, FL (US); E. Allan EIKMAN, Tampa, FL (US)

(21) Appl. No.: 14/770,635

(22) PCT Filed: Jan. 21, 2013
(86) PCT No.: PCT/US2013/022397

§ 371 Date: Aug. 26, 2015

(51) Int. Cl. A61B 17/132

(52) U.S. Cl. CPC A61B 17/1322; A61B 2019/4842

(57) ABSTRACT

A disposable tourniquet and its method of use is disclosed. The tourniquet is in the form of a band formed of a thin flexible, non-stretchable material and is arranged to be wrapped around the patient's limb, tightened and locked in place to reduce circulation. The tourniquet includes a removable portion which includes indicia bearing information, e.g., instructions for use, promotional materials, etc. The removable portion may be in the form of an envelope or pouch for holding some item therein. The removable portion may form a tearable portion of the band or may comprise a panel or patch adhesively secured to the band. The tourniquet also may include at least one usage-evidencing seal.

### CROSS REFERENCE TO RELATED APPLICATIONS

This application is a national stage application that claims the benefit under 35 U.S.C. §371 of International Application No. PCT/US2013/022397 filed on Jan. 21, 2013 entitled DISPOSABLE VENOUS TOURNIQUETS WITH REMOVABLE INDICIA-BEARING PORTION AND METHODS OF USE and whose entire disclosure is incorporated by reference herein.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### BACKGROUND OF THE INVENTION

1. Field of the Invention

This invention relates to generally to medical devices and more particularly to tourniquets, e.g., venous tourniquets which are used during a venipuncture process of withdrawing blood from a limb of a patient, and methods of using the same.

2. Prior Art

The process of venipuncture is eased by the use of a tourniquet to increase tissue pressure to retard venous flow, thereby to facilitate the distention of veins along an extremity such as an arm or leg. By applying a venous tourniquet the vein enlarges, becomes more visible, and the wall is tensioned, helping to facilitate needle entry in the proper location and depth with less prolonged patient discomfort. The tissue pressure achieved is ideally just sufficient to accomplish distension with minimal discomfort, and without interference in arterial flow. Less tissue pressure may be required if the patient is not subject to peripheral venous vasoconstriction, as when the care environment is cool, and venous vasoconstriction influences strive to retain body heat.

A rubber strip is most commonly used as a low cost venous tourniquet, by stretching, then releasing a locked length of strip to apply pressure to the tissue. As the tourniquet is looped and tightened around the extremity, the elastic material narrows and buckles inward as pressure along the arm is focused on less surface area. This property of rubber prevents the tourniquet from realizing the spread profile of pressure which is both effective and comfortable. It is also difficult to estimate the actual pressure being applied when the stretched rubber is released to apply to the extremity. The rubber strip tourniquet is commonly secured with a tuck hitch which may slip and snap back, and uses a variable, uncertain length of strip, further impairing estimation of the actual tissue pressure being applied. Upon securing the tourniquet, the patient often feels a pinching sensation located where the band intersects itself, or due to narrowing of the band with tightening. In addition, the application of the tourniquet typically may move the extremity as the tourniquet is pulled away from the extremity and with over tightening in order to gain enough slack to secure a tuck hitch. Then as the process is finished, the patient may experience additional pinching and hair pull as the skin is stretched along the extremity, and at the tuck. With application of the tourniquet complete, the person applying the tourniquet does not have a direct way of feeling or otherwise appreciating the tension of the tourniquet, no more than necessary to achieve distension. Even experienced users may not consistently provide the appropriate amount of tension. Although under-tension is evident because or poor venous distension, a user may consistently over tension the tourniquet failing to learn to adjust tension appropriately.

Several improvements over the rubber strip have been disclosed in the patent literature. For example, U.S. Pat. No. 3,930,506 (Overand) discloses a disposable tourniquet with an adhesive strip protected by a liner. The feature eliminates the need to tie a tuck hitch, but the release liner has to be removed producing waste material. Moreover, the device is elastic so that it becomes narrower and buckles inward as tension increases. Further still, it has no means to indicate the level of tension.

U.S. Pat. No. 5,219,356 (Harreld) discloses a device which addresses the problem of producing additional waste material by folding over a portion of the tourniquet with the release liner attached. Like the Overand device, the device of Harreld is made with an elastic material and has no tension indication. In addition, the device has no grip for a user's second hand to hold during application, such that the second end of the tourniquet must pass over and rest on top of the fingers (holding the first end in place) before contacting the pressure sensitive adhesive. When the fingers are removed the tension changes unpredictably.

U.S. Published Patent Publication 2007/0250109 (Kerstein et al.) discloses a tourniquet which attempts to solve that problem by introducing a grip region and an aperture or through hole for the other end. However, the feature of Kerstein et al. for achieving that end leads to a several problems. In particular, as the tourniquet is tightened the aperture becomes a pinch point and skin will push through the location. In addition, like the other devices described above the device of Kerstein et al. has no tension indication.

U.S. Pat. No. 5,540,714 (Lundberg et al.) is directed to a disposable tourniquet which prevents slippage making it easier to knot and results in less pulling of skin and hair, but the tourniquet's band still narrows as tension is increased. Moreover, as is the case of the other prior art described above, the device of Lundberg et al. doesn't provide any tension indication.

Several patents do, however, disclose tension indication, e.g., U.S. Pat. No. 3,613,679 (Bijou), U.S. Pat. No. 5,195,950 (Delannoy), U.S. Pat. No. 5,779,659 (Allen) and U.S. Pat. No. 5,894,032 (Green et al.). But these prior art devices all necessitate the use of elastic material as the body of the device in order to function. U.S. Published Patent Application 2009/0062843 (Heston) discloses a device that adds a resilient section to the body of the band. But again elasticity in the body of the device is necessary. U.S. Pat. No. 6,149,168 (Sato) provides tension indication by means of a spring which for the purpose of a disposable tourniquet would be cost prohibitive. Comfort features are also described, but the use of the holding ring at the point of intersection between the first and second end of the device would result in skin pushing through the location when tension is applied. A pulley feature is also described, but it lacks a sleeve. Thus, the device cannot slide across the extremity and tissue may be uncomfortably torqued.

U.S. Pat. No. 7,842,067 (Esposito) introduces a sleeve. But there is no direct tension indication other than remembering the number of turns of a rod. Moreover, the features of the Esposito tourniquet constitute a level of complexity which would not be conducive to single use disposable tourniquets.

U.S. Published Patent Application 2008/0177159-A1 (Gavriely) discloses a tourniquet used to stop arterial blood loss that includes a temperature monitor to contribute to diagnostics including to warning signals that usage time is about to expire. There is no provision to indicate ambient temperature to indicate a possible state of peripheral venoconstriction prior to application of a tourniquet to facilitate venipuncture.

To summarize, all of the venous tourniquets described in the prior art suffer from one or more the following disadvantages: pinching of the skin (including where the band intersects itself); pinching and pulling of hair including along the back of the extremity, imprecise or no tension control by the user; use of elastic material which narrows and buckles when stretched, focusing more pressure on less surface; and being cost prohibitive as a disposable tourniquet. Moreover, such devices do not include any ambient temperature feedback to indicate if conditions are ideal for venous distension and typically have excess material which must be removed prior to use or additional behaviors during operation preventing convenient application.

In our PCT Application PCT/US2011/066010 there is disclosed and claimed tourniquets and methods of use which overcome the disadvantages of the prior art. While the invention of that application is generally suitable for its intended purposes, we have determined that an improvement of those tourniquets and the methods of use would be provided if each of the tourniquets includes a removable portion including indicia, such as instructions for use and, if desired, promotional materials, which could be given to the patient on whom the tourniquet is used. That improvement constitutes the basis of the subject invention.

### SUMMARY OF THE INVENTION

In accordance with one aspect of this invention there is provided a disposable tourniquet arranged to be wrapped around a patient's limb and tightened to reduce circulation. The tourniquet comprises a band formed of a thin flexible, non-stretchable (inelastic) material. The band is includes a removable portion bearing indicia, e.g., information regarding usage of the tourniquet, advertising or promotional materials, premiums, etc. In accordance with one preferred embodiment of the invention the tourniquet is constructed to control the amount of tension applied to the band when it is tightened about the limb of the patient.

In accordance with another aspect of this invention there is provided a method of applying a tourniquet constructed like that described above onto the limb of a patient, with the removable portion being removed either before or after the band is tightened about the limb of the patient so that the patient can be given the removed portion to provide usage instructions.

### DESCRIPTION OF THE DRAWING

FIG. 1A is a plan view of one exemplary tourniquet constructed in accordance with the invention of our aforementioned PCT Application PCT/US2011/066010;

FIG. 1B is a reduced isometric view of a stack of exemplary tourniquets, each of which is constructed like the tourniquet shown in FIG. 1B;

FIG. 1C is a reduced isometric view of the tourniquet of FIG. 1A, but shown being unfolded from a compact folded storage configuration;

FIG. 1D is an isometric view, like that of FIG. 1 C but showing the tourniquet being unfolded from an even more compact folded storage configuration;

FIG. 1E is an enlarged isometric view of the tourniquets of FIG. 1A shown in the state where it is ready to be tightened about the limb, e.g., arm, of a patient (the limb not being shown in this figure);

FIG. 2A is a plan view of an alternative exemplary embodiment of a tourniquet constructed in accordance with the invention of our aforementioned copending PCT Application;

FIG. 2B is an illustration taken from a transverse direction showing how the tourniquet of FIG. 2A is disposed about the arm of a patient;

FIG. 3 is an isometric view of an alternative embodiment of a tourniquet constructed in accordance with the invention of our aforementioned PCT Application, with that alternative embodiment being shown with a plurality of folds provided in the main strip parallel to the main strip and with portions of the tourniquet wrapped around each other;

FIG. 4A is an isometric view of still another alternative exemplary embodiment of a tourniquet constructed in accordance our aforementioned PCT Application, with that embodiment shown in the assembled state where it is ready to be wrapped about the limb, e.g., arm, of a patient (the limb not being shown in this figure);

FIG. 4B is a plan view of the cut sheet of material used to make the tourniquet of FIG. 4A when that sheet is laid flat and ready to be folded and assembled into the tourniquet;

FIG. 5 is an isometric view showing still another exemplary alternative embodiment of the tourniquets constructed in accordance our aforementioned PCT Application, with the tourniquets of FIG. 5 making use of tab that can be used in place of the tabs of the tourniquets shown in FIGS. 1, 2 and 3;

FIG. 6 is a plan view of one exemplary embodiment of a tourniquet constructed in accordance with this invention, wherein the tourniquet is similar to the tourniquet of FIG. 1A, but includes a removable portion bearing indicia thereon;

FIG. 7 is a plan view of another exemplary embodiment of a tourniquet constructed in accordance with this invention wherein the tourniquet is similar to the tourniquet of FIG. 1A, but includes an alternative exemplary removable portion bearing indicia thereon;

FIG. 8 is a plan view of still another exemplary embodiment of a tourniquet constructed in accordance with this invention wherein the tourniquet is similar to the tourniquet of FIG. 1A, but includes another alternative exemplary removable portion bearing indicia thereon;

FIG. 9 is a plan view of still another exemplary embodiment of a tourniquet constructed in accordance with this invention wherein the tourniquet is similar to the tourniquet of FIG. 1A, but includes another alternative exemplary removable portion, i.e., a pouch bearing indicia thereon; and

FIG. 10 is a plan view of still another exemplary embodiment of a tourniquet constructed in accordance with this invention wherein the tourniquet is similar to the tourniquet of FIG. 9, but includes an alternative exemplary pouch bearing indicia thereon.

### DETAILED DESCRIPTION OF THE PREFERRED EMBODIMENT

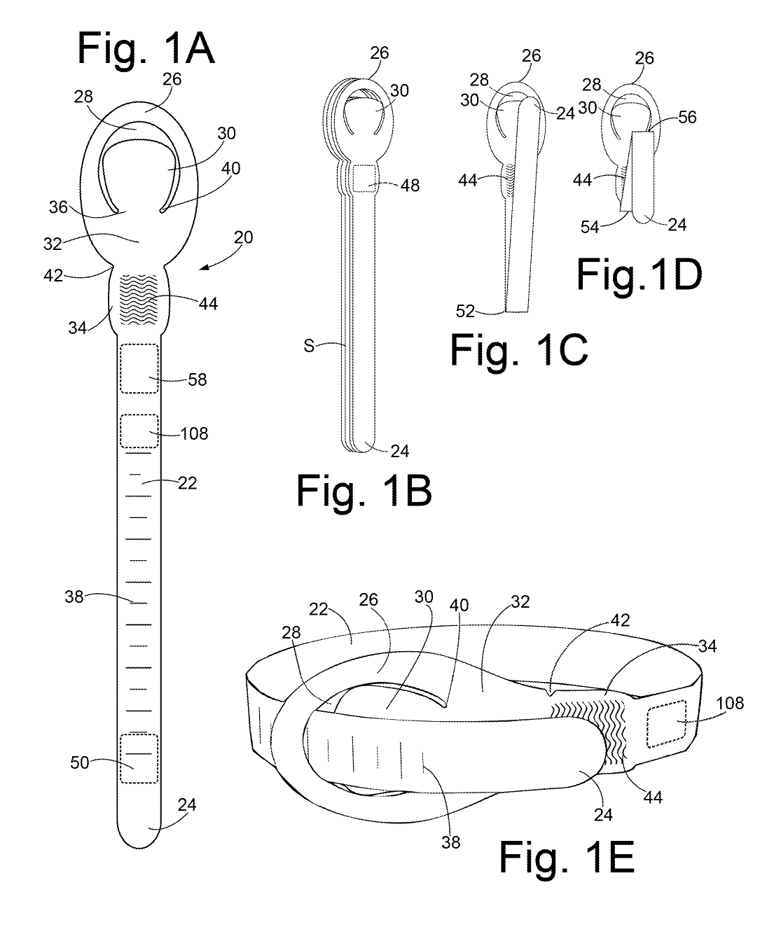

Referring now to the various figures of the drawing wherein like reference characters refer to like parts, there is shown in FIG. 1A one exemplary embodiment of a tourniquet 20 constructed in accordance with our aforementioned PCT Application.

It must be pointed out at this juncture that the word “exemplary” is used herein to mean “serving as an example, instance, or illustration.” Any configuration or design described herein as “exemplary” is not necessarily to be construed as preferred or advantageous over other configurations or designs. Furthermore, use of the words “present invention” or “instant invention” is used herein is meant to include alternative embodiments to the disclosed exemplary embodiments that one of ordinary skill in the art understands. Thus, while some of the embodiments of the invention will be described more fully hereinafter with reference to the accompanying drawings, in which exemplary embodiments are shown, this invention may, however, be embodied in many different forms and should not be construed as limited to the embodiments set forth herein. All statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Moreover, the functions of the various elements shown in the figures may be provided through the use of materials that may vary in shape, attachment, size, and other physical features.

Thus as can be seen in FIG. 1A the tourniquet basically comprises a band which is preferably cut from a single sheet of flexible, inelastic (non-stretchable) plastic, such as non-woven olefin fiber or flashspun high density polyethylene such as sold under the trademark TYVEK®. Other suitable non-stretchable materials are also contemplated, such as but not limited to high strength cellulose and composites. The use of an inelastic band which is formed of a relatively smooth material prevents the tourniquet band from narrowing when tension is applied and also prevents the pulling of patient's hair underlying the band. Moreover, the simplicity of the single sheet construction and use of cost effective materials making up the tourniquet promotes economically viable disposal.

The band includes a main strap portion 22 which is of a predetermined and relatively narrow width, e.g., approximately 1 inch (2.54 cm) and terminates in a rounded (e.g., semi-circular) free end 24. A plurality of indicia, e.g., lines, 38 are equidistantly spaced along the strap portion 22. These lines 38 serve as a means to aid the user in determining the amount of tension applied by the tourniquet when it is tightened (as will be described later).

The tourniquet includes a set of features which will be described later which promote comfort, proper and consistent tensioning, and rapid application of the tourniquet. To that end, the opposite end of the strap portion 22 from the free end 24 is in the form of an enlarged tab 26 having a through hole 28 into which a tongue 30 projects. The hole 28 is arranged to receive the free end 24 of the strap portion as shown clearly in FIG. 1E when the tourniquet is placed about the limb of a patient for use. The free end of the strap portion can then be pulled by the user, while the tab portion is pulled or held in place to tighten the tourniquet around the limb, e.g., arm, of the patient.

When the tourniquet has been tightened to the desired level of tension it can be locked into place by releasable securing means. That means is in the form of a pressure sensitive adhesive patch arranged to be releasably secured to the undersurface of the strap portion 22 brought into engagement with it. In accordance with one preferred aspect of this invention the pressure sensitive adhesive patch 44 has approximately 50 oz./in. strength. The patch serves as a locking zone which is arranged to be secured to portion of the underside of the tourniquet's strap portion 22 when the tourniquet is tightened to the desired level of tension. The adhesive patch (locking zone) is preferably surrounded by a blank or clear border area, which serves to keep the patient's hair away from the adhesive. It should be noted that the subject invention contemplates that other releasable locking means, e.g., cooperating multi-hook and multi-loop fastening components (like those sold under the trademark VELCRO®), may be used in lieu of the adhesive patch. Other releasable securing means can be used as well.

As can be seen in FIG. 1E the tongue 30 is wider than the width of the main strap portion 22, such that the tongue flanks the main strap portion 22 when the tourniquet is applied (e.g., wrapped about the arm of the patient). The tab includes a base portion 32 interconnecting it to an intermediate portion 34. The intermediate portion 34 includes the adhesive patch area that forms a locking zone 44 for the tourniquet. The base portion of the tab is wider than the tab itself so as to more evenly distribute tension as the tab is pulled. As the free end 24 of the strap portion 22 is pulled across the tab, the intersection of tension between the tab 26 and the free end 24 is located at the base 32, while the strap portion 22 intersects with the tongue 30. The tongue allows the strap portion 22 to slide until the amount of tension that is on the tourniquet is such that friction shifts the tongue towards the base 32 before the patient's skin underlying the tourniquet is pinched. In particular, as the tongue is shifted forward it begins to crumple into a crumple zone 36, which is located towards the bottom region of the tongue. The crumple zone is the narrower part of the tongue for the purpose of gaining the initial traction against the main strap portion 22. Then, as tension is increased the zone expands (propagates) through the rest of the tongue 30. The behavior of the crumpling of the tongue happens in place of crumpling of the underlying skin of the patient, thereby alleviating pinch. The increase in tension throughout the process can be clearly measured by comparing the indicia 38 along the strap portion 22 against the cut 42 or 40.

As will be appreciated by those skilled in the art, although the material used in fabrication of the tourniquet 20 is strong in tensile strength, acute angles of cut can introduce weak points which can tear when tension is applied. Thus, as a general guideline the diameter of the cuts in the tourniquet should be greater than 0.125 inch (3.2 mm). To that end, the cut 40 which is located at the interface of the base 32 and the tongue 30, is at least 0.125 inch (3.2 mm) in diameter to prevent premature tearing thereat. The cut 42 which is located at the intersection of the base 32 and the intermediate section 34 is also similarly constructed to help prevent tears. The cut 42 serves another distinct purpose by separating the base of the tourniquet from the locking zone 44. It is important to note that the greater the distance between the base 32 and locking zone 44 allows for more distinct motions between the act of tensioning the strap portion 22 and the act of locking the strap portion due to curvature of the limb (e.g., arm).

As mentioned above the tourniquet includes indicia 38 on its band to enable the user to determine the amount of tension applied by the tourniquet when it is tightened. This is accomplished by the user visually referring to those measuring lines 38 with reference to the cuts 40 and/or 42 or to the base 32.

The tourniquet may also include means to indicate the temperature of the the ambient surroundings. That means may comprise temperature sensitive liquid crystals 108 (FIGS. 1A and 1E) located in an area on the tourniquet so as to be readily visible when the tourniquet is about to be placed, to verify that patient is not likely to be in a state of peripheral venous constriction to conserve heat.

The tourniquet may be provided in the form of a stack S of identical tourniquets 20, such as shown in FIG. 1B. The tourniquets of that stack are releasably secured to one another. Thus, when a pressure sensitive adhesive is used for the locking zone 44 as described above, the tourniquets include a release liner. The release liner can be provided in one of three locations, depending on how the tourniquets are provided. For example, if a plurality of tourniquets constructed like discussed above are provided in a series face down to form the stack 44 like shown in FIG. 1B, a release liner patch 48 is located on the underside of each tourniquet so that it will cover the locking zone adhesive patch 44 on the upper side of the tourniquet located below it in the stack. Thus, the liner patch 48 on each tourniquet serves to releasably engage the adhesive of the locking zone of the underlying tourniquet, thereby releasably securing the tourniquets of the stack to one another so that the topmost tourniquet can be easily pulled from the stack for use.

The tourniquets shown in FIGS. 1C and 1D are constructed so they may be folded to render them more compact for storage until they are ready for use. In particular, FIG. 1C shows the tourniquet 20, with its main strap portion 22 folded once lengthwise in order to hold the tourniquet in a compact state until ready for use. When the tourniquet is so arranged, another release liner patch 50 is used to protect the adhesive of the locking zone 44. In particular, the release liner patch 50 is located on the upper surface of the strap portion 22 adjacent its free end 24 so that when the strap is folded along a central fold line 52, such as shown in FIG. 1C, the release liner will be disposed over the adhesive making up the locking zone 44. The tourniquet 20 can be folded into an even smaller configuration (footprint) as shown in FIG. 1D, by folding its strap portion 22 along fold lines 54 and 56. In that case the tourniquet includes still another release liner patch 58, which is located on the upper surface of the strap portion 22 adjacent to the locking zone as shown in FIG. 1A for engaging the adhesive patch 44.

The use of the tourniquet 20 will now be described with reference to FIG. 1E. In particular, the tourniquet the tab 26 is grasped by the user between his/her thumb and forefinger and with the locking zone facing up. The main strap portion 22 is then loosely wrapped around the limb of the patient. With the opposing hand, the free end 24 of the strap portion 22 is guided through the opening 28 to form a loop. The band portion 22 is aligned to overlap itself, and the tongue 30, which is wider than band portion, so that the extension of tongue 30 on either side of the side edges of the band portion 22 is defined as a flange of tongue. The tourniquet is then adjusted approximately to the preferred position on the patient's limb, e.g., 1-3 cm superior (cephalad) to the venipuncture target, approximately centered in an axial plane. With the tourniquet in position the end of the tab 26 is held by one hand of the user. The band portion 22 is held by the user's other hand at any convenient point therealong, considering the diameter of the patient's limb on which the tourniquet is to be disposed. The grasped tab 26 and the grasped band portion are then pulled in opposite directions while approximately tangent to the curve of the patient's limb, so as not to prematurely intersect the adhesive patch 44 of the locking zone.

As should be appreciated by those skilled in the art at any time the tourniquet 20 may be temporarily positioned in approximate position with minimal tension, not impeding venous flow, while the user attends to patient needs, such as adjustments of blankets to improve body warmth to meet minimum indicated by an integral ambient temperature indicator, e.g., temperature sensitive liquid crystals, which are provided on the tourniquet as described earlier.

At this point in the process at least two options of usage are possible. In the first option: the tab 26 and band portion 22 are pulled until the adequate tension is reached. The user, by experience with the feel of the relatively inelastic band against pull on tab 26, learns the approximate additional tensioning required as indicated by the metered lines (the indicia 38) on the main strap of the tourniquet, advancing from lightly closed to fully tensioned. The amount of tension depends on multiple factors, including inherent tissue turgor, the diameter of the extremity, and the location of the tourniquet on the extremity. When the desired amount of tension is applied, the tourniquet is ready to be locked in place to maintain that tension. To that end, the user visually verifies that tension is maintained while he/she pulls the free end 24 of the band portion in toward parallel, to intersect the curve of the patient's limb at the location of the locking zone 44. The pressure of the underside of the band portion 22 on the adhesive of the locking zone 44 causes that adhesive to releasably secure the tourniquet in place about the limb of the patient with the desired degree of tightness since the adhesive is sufficiently strong in shear to hold such tension. In case any further adjustment of the tourniquet is desired, the free end 24 of the band portion 22 can be lifted off of the adhesive to unlock the tourniquet and the tourniquet readjusted as needed, e.g., the processes easily repeated.

In the second option of usage: the main strap is observed to be aligned with the axis of the tongue, with tongue flanges approximately symmetrically extending lateral, under the overlying strap portion 22. The user then holds the strap portion 22 close to loop exit, and temporarily presses with his/her thumb on base 32 just beyond base of tongue to gently anchor the closed loop.

It is anticipated that any user of the subject tourniquet will learn by experience to estimate the tissue turgor by the resistance to thumb pressure, and to detect deformity of tissue short of pinch. The user can note the position of the tourniquet using the indicia 38, so that by experience he/she learns the approximate additional tensioning required as indicated by that indicia. This tension depends on multiple factors, including inherent tissue turgor, the diameter of the extremity, and the location of the tourniquet on the limb. The user then continues to anchor the tourniquet in position on the patient's limb by pressing on the base 32 with his/her thumb and releasing his/her grasp on the tab 26 and shifting his/her thumb to a lateral flange of the tongue (the portions of the tongue which lie outside the edges of the overlying band portion 22). The user then gently depresses the flange to maintain anchor pressure. This enables additional pulling of the band with the user's now free hand along the tangent, remaining approximately aligned in axial plane. The user then adjusts the pull by sliding the band portion 22 in either direction along anchored tongue. The flange of the tongue 30 is then depressed as necessary to depress the underlying tissue and to straighten the tongue to minimize any potential to pinch the patient's skin at the tongue. When sufficient pressure is realized to increase tissue pressure to retard venous flow return, the user then angles the free end inward toward parallel to the curvature of patient's limb. The strap portion 22 is pressed down onto the locking zone 44 to lock the tourniquet in place at the set level of tightness. If any further adjustment is necessary the free end 24 can be lifted up to release the band section from the adhesive patch 44 of the locking zone and the processes easily repeated.

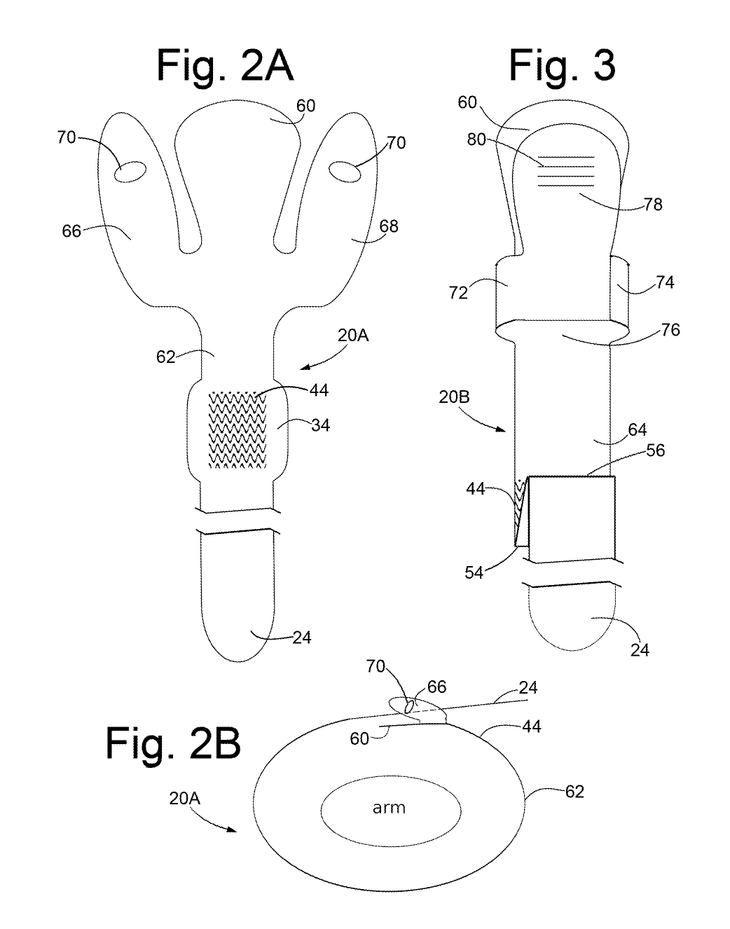

FIGS. 2A and 3 illustrate additional embodiments 20A and 20B of the tourniquet. Like the embodiment of FIGS. 1A-1E both of the embodiments 20A and 20B are cut from a single sheet of the same material as embodiment 20, and have similar main strap features. In the interest of brevity the common features that the tourniquets 20A and 20B share with the tourniquet 20 will be given the same reference numbers and a description of their construction, arrangement and operation will not be reiterated.

The tourniquet 20A is cut from a sheet of material into the shape shown in FIG. 2A. As can be seen the tourniquet 20A includes a tongue 60, which like the tongue 30 of the tourniquet 20 prevents pinching of the skin of the patient when the tourniquet is applied. Both of the embodiments 20A and 20B include main strap portions. In particular, the tourniquet 20A includes a main strap portion 62, while the tourniquet 20B includes a main strap portion 64. The main strap portions 62 and 64 of those tourniquets are shown concatenated and their measuring lines (indicia 38) are omitted in the interest of drawing simplicity. The critical difference in these embodiments is in their tabs. In particular, the tourniquet 20A includes a pair of symmetrical tabs 66 and 68, each including a hole 70 therein. Such an arrangement does not need any feed through during application. Both of the tabs 66 and 68 can be grabbed, with the thumb and forefinger of the user. The user is able to sense that that the tabs are held firmly by the sensation of his/her fingers against one another through the holes 70. FIG. 2B illustrates that when the tabs are grabbed they are face to face such that they project generally perpendicularly to the patient's limb's (e.g., arm's) axial plane.

The tourniquet of FIG. 3 also has a tongue 60 (constructed like the tongue 60 of the tourniquet 20A) to prevent pinch. The tourniquet 20B also includes a pair of tabs 72 and 74. The tabs 72 and 74 project outward from the sides of the tongue 60 in a similar manner to the tabs 66 and 68, but do not include holes therein. FIG. 3 illustrates the tabs 72 when folded over during fabrication, with the underside of tab 72 adhering to the topside of tab 74 by an interposed adhesive (not shown), and with both tabs lying generally parallel to the main strap 44. By combining the tabs 72 and 74 as just described a through hole 76 is formed in the tourniquet through which the free end of the main strap portion 64 can be extended (fed through). With the tabs 72 and 74 joined as just described a finger grip portion 78 results. The portion 78 includes finger grip lines or indicia 80, indicating that the portions of the conjoined tabs which are to be grabbed with the thumb and forefinger of the user to tighten the tourniquet about the arm of the patient.

The use and operation of the tourniquets of FIGS. 2 and 3 is nearly identical to that of the tourniquet of FIG. 1. One difference with the tourniquet 20A of FIG. 2 is that a single one of the tabs 66 and 68 can be grabbed and held in place against the patient's limb as tension is applied. FIG. 2B shows the tourniquet 20A in place ready to be tightened about the arm of the patient.

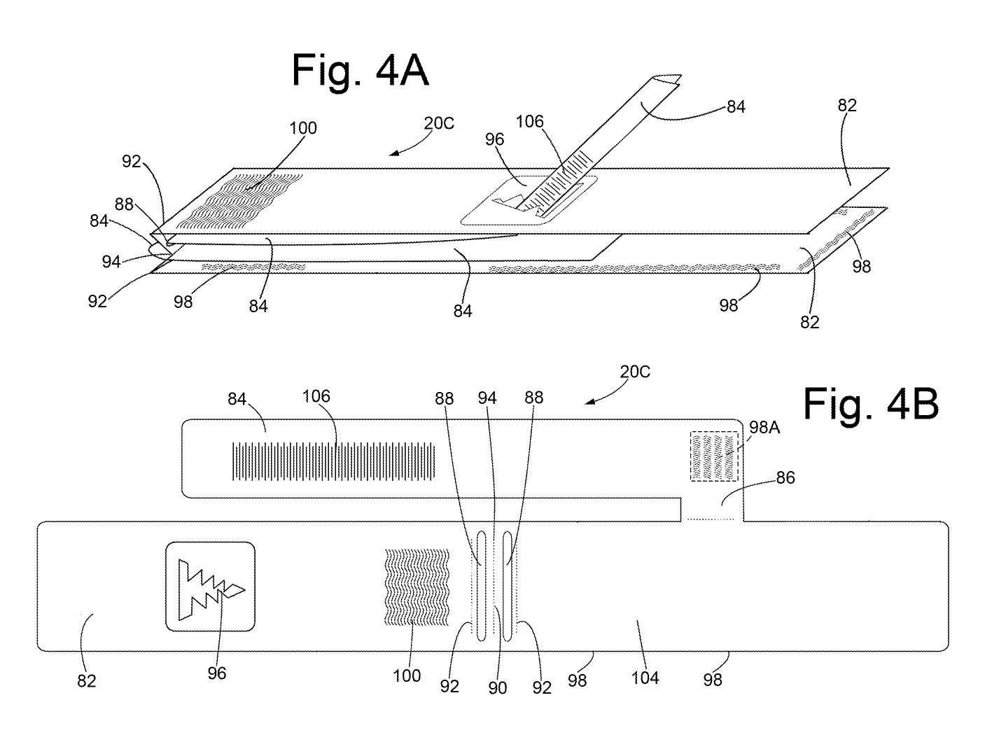

FIGS. 4A-4B illustrate still another exemplary embodiment of a tourniquet 20C constructed in accordance with our aforementioned copending PCT Application. The tourniquet 20C makes use of an alternative approach to prevent tissue pinching and to provide overall comfort and ensure consistent application. Like the other embodiments described above, the tourniquet 20C is cut from a single sheet of material into the shape shown in FIG. 4B. The tourniquet 20C is constructed in a manner somewhat like a conventional blood pressure cuff. To that end, as best seen in FIG. 4B the tourniquet basically comprises a band portion 82 in the form of an elongated web and a strip-like extension 84. The extension 84 is an elongated body which extends along the band portion and is connected to it by an intermediate portion 86. The extension is arranged to be folded (as will be described later) to form a pull strap (FIG. 4A) which extends out of a port 96 of the tourniquet. The pull strap 84 is arranged to be pulled by the user to tighten the tourniquet about the limb of the patient after the tourniquet is wrapped about that limb. To that end, the pull strap cooperates with plural slots in the band portion 82 to form an internal pulley arrangement. In particular, the band portion 82 includes a pair of transverse slots 88 which extend across a substantial portion of the width of the band portion 82 at the middle thereof.

The band portion is arranged to be folded along an inner transverse fold line 90 and a pair of outer transverse fold lines 92 located adjacent the slots so that the slots overlie each other to form a passageway through which the pull strap 84 may pass to form the pulley arrangement. To that end, the extension 84 is folded along its longitudinal axis to render it narrower. Then the folded extension is folded toward the band portion 82 along a fold line at the interface of the intermediate portion 86 and the band portion 82. A patch of adhesive 98A is located on the under surface (the surface opposite the surface shown in FIG. 4B) of the extension 84 adjacent the intermediate section 86 so that when the extension is folded to form the pull strap the adhesive patch 98A engages an opposed surface of the band portion 82 to form an anchor for the pull strap. The free end of the extension 84 is then passed through the slots 88 and out of a hole in the band portion. That hole forms the heretofore identified port 96. The port 96 preferably is in the form of a cleat lock. Once the free end of the pull strap has been extended through the cleat lock port, the two opposite ends of the band portion 82 are juxtaposed as shown in FIG. 4A so that they confront each other. As shown in FIG. 4A the inner surface of the band portion 82 (i.e., the surface on which the adhesive patch 98A is located) includes a plurality of lines of adhesive 98 along its periphery. Those adhesive lines secure the two confronting juxtaposed portions of the band portion 22 together and thus complete the tourniquet.

Like the other embodiments described heretofore the tourniquet 20C includes metering indicia 106 on it to provide an indication of the amount of tension applied. In particular, the metering indicia are located on the end portion of the pull strap 84 on the surface shown in FIG. 4B.

When the tourniquet is ready for use it can be readily formed into a sleeve or cuff to be wrapped about the limb of the patient. In order to hold the tourniquet in that configuration, the tourniquet includes releasably securable means in the form of a lock and cooperating locking zone. In the exemplary embodiment shown the lock preferably comprises a patch 100 of a pressure sensitive adhesive like used in the other embodiments 20, 20A and 20B. The locking zone (not shown), is on the undersurface of the tourniquet at the opposite end than the patch 100. Thus, when the tourniquet is wrapped in place about the limb of the patient the patch 100 on the free (outer) end of the tourniquet is disposed opposite the locking zone, and is pressed into place, thereby forming the tourniquet into a sleeve or cuff.

Tightening of the sleeve or cuff is achieved by the user placing his/her thumb and forefinger on top of the cleat lock at the outlet port 96 and holding it down. The free end of the pull strap 84 is then pulled tangentially from the cleat by the user's other hand while the cleat lock is held. The pulley arrangement provides mechanical advantage to increase the tension. This action causes a crumple zone portion 104 (FIG. 4B) of the band portion 82 to crumple, thereby shortening the length of the band portion so that the sleeve or cuff tightens around the patient's limb. The pulling of the pull strap out of the cuff causes the metering indicia 106 along the pull strap 84 to be revealed as the strap is pulled, thereby indicating the amount of tension applied. Once the necessary tension is reached the pull strap is pulled at an angle to engage the cleat lock and thereby releasably lock the tourniquet in place at the desired amount of tension. Alternatively, an optional pressure sensitive adhesive lock (not shown) can be provided on the top of the cuff adjacent the exit port. In such a case, after the pull strap is pulled to establish the desired amount of tension the pull strap is pressed parallel into engagement with the pressure sensitive adhesive lock to releasably lock the tourniquet in place at that level of tension.

It should be pointed out at this juncture that alternative means can be used to releasably secure the tourniquet about the limb of the patient. One such releasably securable means comprises VELCRO® multi-hook and multi-loop fasteners.

As will be appreciated by those skilled in the art, when the tourniquet 20C is in place on the limb of the patient the sleeve or cuff encloses the pull strap 84 preventing it from coming in direct contact with the patient's limb, and allowing the pull strap to move freely across the inside of the cuff through the slots 86 and 88 and out of the port 96.

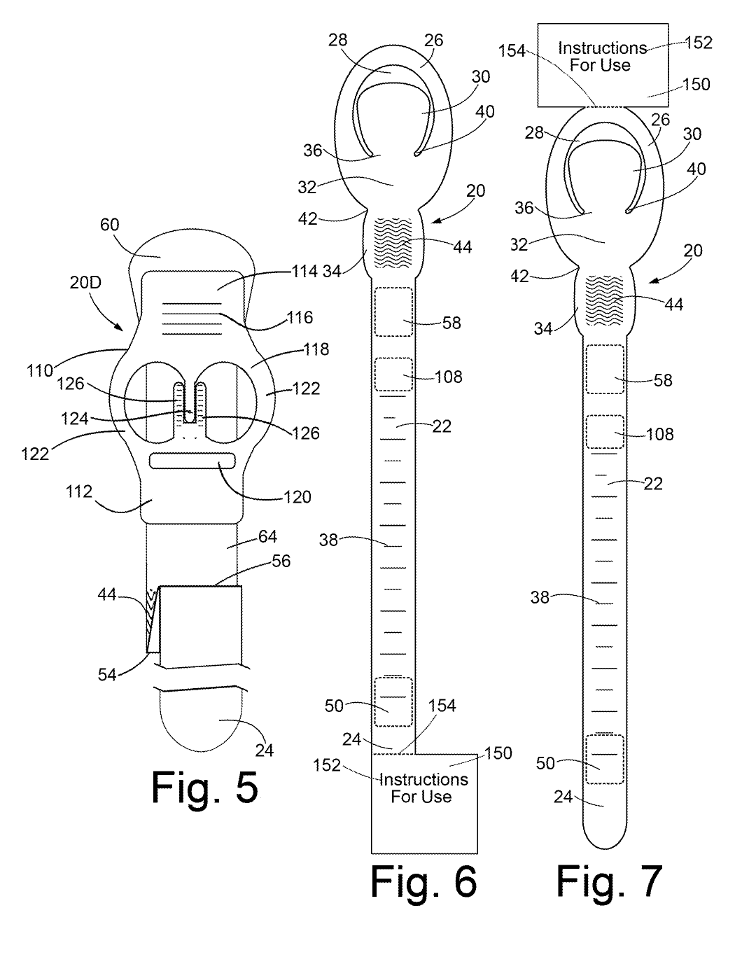

Turning now to FIG. 5, yet another exemplary embodiment of the tourniquet 20D is shown and will now be described. The tourniquet 20D is a variant of the tourniquet 20B shown in FIG. 3. Thus, the features of tourniquet 20D that are common to tourniquet 20B will be given the same reference numbers and the details of their construction and operation will not be reiterated in the interest of brevity. As can be seen the tourniquet 20D includes a variant tab 110 which provides feedback to the user showing the amount of tension applied by the tourniquet when it is tightened. The tab 110 basically comprises a body of a relatively rigid plastic having a base portion 112, and an upper portion 114. The base portion 112 is fixedly secured to a portion of the band portion 64 below the tongue 60. The upper portion of the tab is not secured to the tongue and includes a finger grip zone 116 at the upper end of the tab and a central portion 118 located between the finger grip zone and the base portion. A slot 120 is located in the central portion immediately above the base portion 112. The central portion is arranged to stretch upon the application of tension to the tab. To that end, it includes a pair of flex legs 122 that bridge the central portion of the tab from the finger grip zone to the base portion. The legs 122 are relatively thin so that they can flex when tension is applied to the tab (as will be described shortly), whereupon the central portion of the tab stretches. A tension indicator, in the form of a finger 124, projects downward from the finger grip zone between a pair of fingers 126 that project upward from the base portion. The fingers 126 include indicia along their length, which in cooperation with the free end of the finger 124 serve to provide an indication of the amount of tension applied by the tourniquet 20D when it is tightened.

In use the free end portion of the tourniquet is wrapped around the limb of the patient and brought over the upper surface of the tongue 60, under the upper and intermediate portions of the tab 110 and out of the slot 120. In order to tighten the tourniquet the user grasps the finger grip zone 116 of the tab between his/her thumb and forefinger of one hand while the pulling the free end 24 of the band 64 away from the slot 120. This action applies a force to the leading edge of the slot, whereupon the tab 110 begins to stretch at its central portion 118, i.e., its legs 122 flex and deform, so that the tip of the indicating finger 124 moves to a new position with respect to the indicia on the fingers 126, thereby indicating the amount of tension applied. The user can apply as much tension as desired. Once that level of tension is achieved (which is indicated by the indicating finger and the associated indicia), the free end 24 of the band 64 can be brought into engagement with the adhesive patch 44 on the band to releasably lock the tourniquet in place.

It should be noted that other stretchable arrangements are contemplated for the tab, e.g., other shaped flex legs can be used, the slot can have a side opening so that the free end of the band 22 can be inserted into it from a lateral direction, etc. Furthermore, other tension indicators can be used. For example, it is contemplated that the band 64 include a plurality of accordion-like pleats, with respective patches of adhesive interposed between immediately adjacent pleats, and with each succeeding patch of adhesive being of greater tensile strength than the preceding patch. Thus, the amount of tension applied will be indicated by the number of pleats that have opened when tension has been applied to the band sufficient to overcome the tensile strength of the patch(es) between the accordion pleats.

As should be appreciated from the foregoing the tourniquets as described above exhibit numerous advantages over the prior art. In particular, they include means to prevent pinching and pulling of hair along the extremity. In addition, they provide a form which prevents the body of the tourniquet from narrowing and has means to indicate to the user a useful sense of tensioning. In addition, the tourniquet's construction enables the use of more controlled force in tensioning the tourniquet and makes use of securing means which produce no scrap material during application and which are conducive to accurate tensioning, rapid application, or adjustment. All of those advantages are accomplished by a device whose construction is cost effective and suitable for economic disposal.

Turning now to FIG. 6 there is shown a tourniquet 20 similar to that of FIG. 1A, but including an “indicia-bearing” feature. That feature is in the form of a panel 150 which includes a surface bearing printed indicia 152. Such indicia can be in the form of education, information, entertainment, branding, or other visually perceptible materials. In the exemplary embodiment shown in FIGS. 6 and 7 the panel's indicia 152 is in the form of instructions for use.

As will be described in more detail shortly, the panel 150 is preferably arranged for ready detachment from the tourniquet when the tourniquet is deployed, although detachability of the indicia-bearing panel is not required.

Inclusion of the indicia bearing panel 150 can serve one or more functions. It can provide printed information for the patient, such as instructions on wound care, contact information, follow-up instructions, text or images comprising distraction from pain or entertainment during venipuncture. It can provide device branding that the patient may understand and retain before, during, and after use of the tourniquet. It can provide a token or coupon for the patient to use in exchange for value, e.g., a coupon for a pharmaceutical purchase, or other object or service of value. It can provide a means to enforce efficient compliance with a procedure check list that includes dispensing the indicia bearing portion for safety, hygiene, and authentication purposes. As should be appreciated by those skilled in the art, the indicia provided on the panel can be of any type and for any purpose. Thus, the examples given above are merely a few of a myriad of types if indicia that can be included on the panel and thus are not deemed to be limiting.

As mentioned above, the panel 150 is preferably detachable from the band of the tourniquet. To that end, the panel may form an integral part of the tourniquet, e.g., it can be formed of the same material making up the band, but is releasably secured to the by a breakable or frangible line, e.g., perforated, line 154. The perforated line 154 can be produced during the manufacture of the tourniquet, or thereafter.

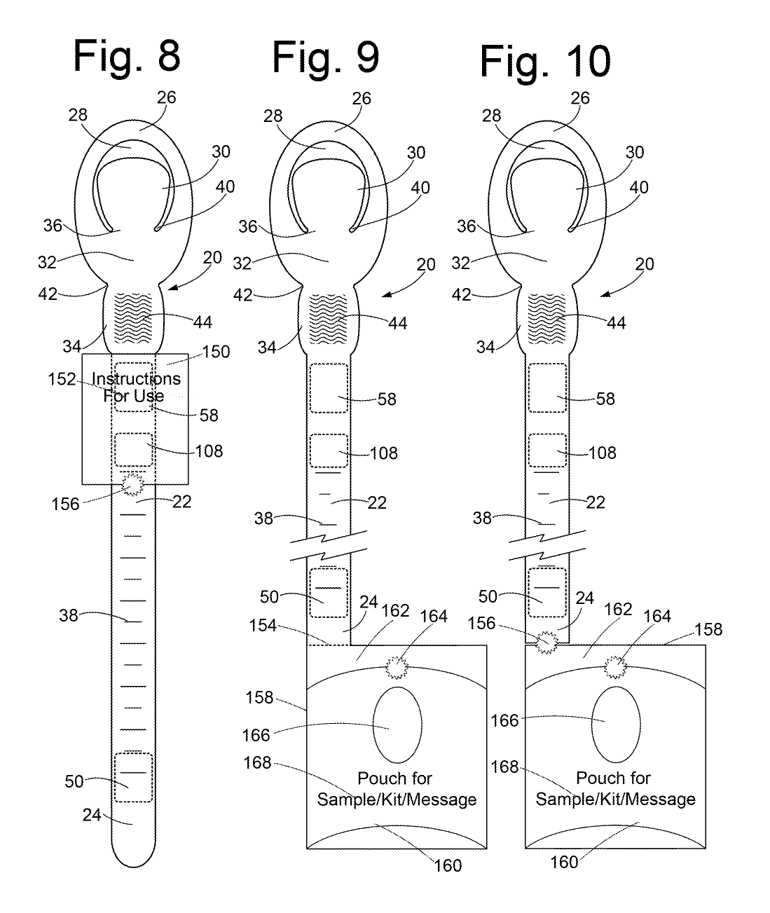

The detachable panel 150 may be located at the free end portion 24 of the tourniquet, like shown in FIG. 6, or may be located at the tab portion 26, like shown in FIG. 7. Alternatively, the panel may be disposed on the band 22 of the tourniquet over the areas 58 and 108, such as shown in FIG. 8. In order to hold the panel 150 in place thereat, an adhesive star-shaped patch 156 is provided. In particular, a portion of the patch 156 is adhesively secured to the panel 150 and a portion of the patch is adhesively secured to the band 22.

The embodiment of the tourniquet 20 shown in FIG. 9 includes a releasable pouch or envelope 158, which may contain any desired contents to be provided to the user of the tourniquet. For example, the contents of the envelope/pouch may be in the form of printed material, a sample, or a kit of components. Such materials may be inserted into the tourniquet after its manufacture, but before its usage.

The envelope/pouch 158 may be any suitable size, depending upon the contents to be held within it. For example, one typical size has interior dimensions of approximately 2 inches by 3.5 inches to hold printed material comparable in size to a standard business card commonly used in the United States. Package inserts designed to fit with a medicine bottle package are often similar in overall dimensions and can be disposed within the envelope/pouch. The materials within the envelope/pouch may include educational materials, such as patient instructions. Alternatively, the materials may include indicia in the form of text and/or graphics designed to distract or entertain a patient during venipuncture. The indicia bearing materials within the envelope/pouch may be for commercial purposes, e.g., a coupon used for purchase or other purpose, and suitable identifiers or branding information applicable to the tourniquet. Other contents of the envelope pouch may be pharmaceutical samples, e.g., pills held within a blister package. In such a case, the envelope/pouch would have to be correspondingly larger in size. So too, if the materials to be held within the envelope/pouch are somewhat voluminous or thick, e.g., multiply folded package inserts, a correspondingly larger envelope/pouch would be required.

As best seen in FIG. 8 it is contemplated that for some applications at least one surface 160 of the envelope/pouch 158 is curved, or easily deformed to avoid compressing the envelope/pouch's contents and for facilitating its ease of removal from the envelope/pouch. Likewise, a kit (not shown), such as an adhesive dressing or antibiotic designed to be applied to the wound site could be conveniently included in the envelope/pouch. In such a case, that may also require an envelope/pouch with center curve or with a deformable panel to avoid compressing its contents.

To provide access to the interior of the envelope/pouch, it may include a flap 162 to form the mouth to its interior, where its contents are located. Alternatively, the envelope/pouch may include a frangible, e.g., perforated, line (not shown) which may be broken to provide access to the interior of the envelope/pouch. If the envelope/pouch includes a flap 162, like shown in FIG. 9, the flap may be held in its closed state by means of an adhesive patch, e.g., star-shaped patch 164. That patch is constructed like the patch 156 and serves as a breakable seal to prevent accidental displacement of the contents and to provide evidence of tampering, e.g., breakage of the seal indicates prior opening of the envelope/pouch.

At least one surface of the envelope/pouch may include a window or view port 166 of any size and shape to visually disclose that contents of the envelope/pouch. The window/view port may be in the form of a transparent panel or may merely be a hole in one of the panels of the envelope/pouch. The vacancy created by the window/port may also facilitate use of a finger to push the contents out of the pouch.

The envelope/pouch may itself include any type of indicia 168 printed on any of its surfaces. In the exemplary embodiment shown indicia is provided on the front surface of the envelope/pouch adjacent the window 166.

Like the other removable portions of the tourniquet described above with respect to FIGS. 6 -8, the envelope/pouch 158 of each of the tourniquets shown in FIGS. 9 and 10 is arranged to be readily removable from the band of the tourniquet. To that end, in the embodiment shown in FIG. 9, the envelope/pouch 158 is releasably secured to the band 22 by a perforated or frangible line 154. Like the embodiments of FIGS. 6 and 7, the perforated line 154 can be produced during the manufacture of the tourniquet, or thereafter. Alternatively, the envelope/pouch 158 may be a separate component from the band 22, but may be releasably secured to it by an adhesive patch 156 bridging the end portion 24 of the band and the contiguous portion of the envelope/pouch. The adhesive patch 156 may be of the same type as used to hold the panel 150 on the band 22 in the embodiment of FIG. 8.

It should be pointed out at this juncture that any of the tourniquets of this invention may include breakable mechanical seals at various locations, which if broken indicate prior usage of tampering of the tourniquet. Such usage-evidencing seals may be in the form of a material that is functionally weaker in comparison with other portions of the tourniquet, and located so that at least one point in the process of deployment or use of the tourniquet, the seal is irreversibly broken to create visual evidence that the tourniquet or some part thereof has been partially or completely deployed at least once. The placement of the usage-evidencing seal may be chosen so that it is broken early in the process of deployment, before the user's gloved hand has become contaminated due to the subsequent process of the venipuncture.

The mechanical construction of the usage-evidencing seal may be integral to the tourniquet, functioning such as a tear off portion weakened by perforations that the user easily uses to separate part of the tourniquet during use, such as illustrated in FIGS. 6-7. As an alternative, or in addition, the perforations can optionally be bridged by a separate mechanical usage-evidencing seal, such as a breakable patch 156/164. In such a case the patch must be broken while tearing along the perforations. A similar usage-evidencing patch can be incorporated in the folded tourniquet embodiment of FIG. 5. In particular, the usage-evidencing patch can be located so that if the tourniquet is unfolded for use, the patch is broken.

Many other ways to accomplish usage-evidencing sealing of the tourniquet are contemplated. For example, a usage-evidencing seal may be applied to the packaging or wrapping of the tourniquet so it must be broken. The goal of the inclusion of a usage indicating seal is to provide a visual indication that is obvious to the user and potentially to others that the tourniquet has been used at least once. Further, the unsealing is facilitated early in the process of using the device, so the tourniquet itself is more likely to be uncontaminated as long as possible while in place during subsequent steps the venipuncture process. Equally important, the message, kit, or sample dispensed to the patient, whether from a pouch or by a removable part of the device is clean when given to the patient.

It should be pointed out at this juncture that the subject invention does not require that the tourniquets be constructed in accordance with the features of our above mentioned PCT application. Thus, the subject invention contemplates inclusion of removable indicia bearing panels or envelopes/pouches on any type of disposable tourniquet.

As should be appreciated from the foregoing, the “indicia-bearing” panel of the subject invention can provide education, information, entertainment, branding, or other visually perceptible material providing some value to the user. Moreover, the inclusion in the tourniquet of at least one usage-evidencing seal, the fracture of which, signifies use/activation of the tourniquet is of some importance and is thus desirable, albeit not mandatory. For example, it facilitates hygienic care. In this regard, the freshly gloved hand of the phlebotomist can be used to break the usage-evidencing seal to activate the tourniquet, then to conveniently dispense the clean indicia bearing portion of the tourniquet to the patient before proceeding, thereby preventing subsequent contamination of the indicia bearing portion if an equivalent indicia bearing portion separate from the tourniquet is dispensed later in the procedure. The breakable portion of the tourniquet also serves the function of deterring re-use of the tourniquet. In addition, it authenticates the dispensing and use of the tourniquet. Such authentication can serve to prevent multiple billing for a single tourniquet, while simplifying inventory accounting, and assuring that a indicia bearing portion with value, such as a coupon, is not diverted from intended recipient.

Without further elaboration the foregoing will so fully illustrate our invention that others may, by applying current or future knowledge, adopt the same for use under various conditions of service.

We claim:

1. A disposable tourniquet arranged to be wrapped around a patient's limb and tightened to reduce circulation, said tourniquet comprising a band formed of a thin flexible, non-stretchable material arranged to be wrapped around the patient's limb and tightened, said band including a removable portion bearing indicia regarding usage of said tourniquet, said removable portion being releasably secured to said band by a frangible connection.

2. The disposable tourniquet of claim 1 wherein said band is constructed to control the amount of tension applied to the band when it is tightened about the limb of the patient,

3. The disposable tourniquet of claim 1 wherein said removable portion comprises the same material as the material forming said band.

4. The disposable tourniquet of claim 2 wherein said removable portion comprises the same material as the material forming said band.

5. The disposable tourniquet of claim 3 wherein said frangible connection comprises a perforated line in said band.

6. The disposable tourniquet of claim 3 wherein said frangible connection comprises an adhesive member.

7. The disposable tourniquet of claim 2 wherein said removable portion comprises a patch adhesively secured to said band.

8. The disposable tourniquet of claim 7 wherein said patch is adhesively secured to said band by a pressure sensitive adhesive.

9. The disposable tourniquet of claim 1 wherein said removable portion comprises a pouch.

10. The disposable tourniquet of claim 2 wherein said removable portion comprises a pouch.

11. The disposable tourniquet of claim 1 wherein said tourniquet includes a usage-evidencing mechanical seal.

12. The disposable tourniquet of claim 11 wherein said usage-evidencing mechanical seal is frangible.

13. The disposable tourniquet of claim 12 wherein said usage-evidencing mechanical seal forms said frangible connection between said removable portion and said band.

14. A method of applying a tourniquet around a patient's limb to reduce circulation, said tourniquet comprising a band formed of a thin flexible, non-stretchable material and having a removable portion bearing indicia regarding usage of said tourniquet, said method comprising:
deploying said tourniquet by wrapping said band around the limb of the patient so that a desired amount of tension is applied;
locking said band in place when said desired amount of tension is achieved; and
either before or after wrapping said band around the limb of the patient removing said removable portion, whereupon said removable portion may be given to the patient to provide information about the use of said tourniquet.

15. The method of claim 14 wherein said removable portion is removed prior to the wrapping of said band around the limb of the patient.

16. The method of claim 14 wherein said removable portion comprises a pouch.

17. The method of claim 14 wherein said tourniquet includes a usage-evidencing mechanical seal, wherein deployment of said tourniquet breaks said usage-evidencing mechanical seal, thereby providing visual indication of usage of the tourniquet.
